(19) United States
(12) Patent Application Publication
(10) Pub. No.: US 2002/0165133 A1
(43) Pub. Date: Nov. 7, 2002

(54) Use of [gamma-hydroxy-N-methyl-leucine9] cyclosporin a for hair growth

(76) Inventors: Sang-Nyun Kim, Yusong-gu (KR); Ho-Jeong Ahn, Yusong-gu (KR); Chang-Woo Lee, Seo-gu (KR); Jung-Hun Kim, Yusong-gu (KR); Jong-Il Kim, Yusong-gu (KR); Heon-Sik Lee, Yusong-gu (KR); Min-Ho Lee, Yusong-gu (KR); Ho-Song Cho, Seo-gu (KR); Seung-Jin Kim, Yusong-gu (KR); Hong-Soon Park, Yusong-gu (KR)

Correspondence Address: VENABLE, BAETJER, HOWARD AND CIVILETTI, LLP, P.O. BOX 34385, WASHINGTON, DC, 20043-9998, US

(21) Appl. No.: 10/073,021

(22) Filed: Feb. 12, 2002

(30) Foreign Application Priority Data
Feb. 14, 2001 (KR) 2001-7263

(51) Int. Cl. A61K 38/13; A61K 7/06; A61K 7/11

(52) U.S. Cl. 514/9; 424/70.14

(57) ABSTRACT

The present invention discloses a hair growth promoter comprising [γ-hydroxy-N-methyl-L-leucine⁹] cyclosporin A, in which a hydroxy group is added to a γ carbon of N-methyl-L-leucine at No. 9 position in cyclosporin A by metabolic action of a microorganism, as an active ingredient.

### TECHNICAL FIELD

The present invention relates to a hair growth promoter comprising a cyclosporin derivative as an active ingredient. More particularly, the present invention relates to a hair growth promoter comprising [γ-hydroxy-N-methyl-L-leucine⁹] cyclosporin A as an active ingredient.

### BACKGROUND ART

On average, the human scalp contains about 100,000 to 150,000 hairs. Each hair has three main stages of growth: anagen, catagen and telogen, after which the hair falls out. This hair growth cycle is repetitive and the duration of one cycle is different from other cycles, ranging approximately 3 to 6 years. Thus, the average adult normally loses about 50 to 100 hairs every day. In general, alopecia refers to a phenomenon wherein duration of the anagen growth phase is shortened and the percentage of hairs in the catagen and telogen phases increases, whereby the number of lost hairs is increased excessively and abnormally.

There are many theories to explain the loss of hair, including for example, poor blood circulation, excessive functioning of male sex hormone, excessive production and secretion of sebum, deterioration of scalp by peroxides, bacteria, etc., hereditary factors, aging, stress, etc. However, explicit mechanisms have not been revealed. Recently, the population suffering from hair loss is tending to increase, since changing dietary habits and stress imposed on individuals due to modern social environments, etc. has increased. Also, the age of the individuals affected by alopecia is dropping and furthermore, the population of female alopecia sufferers is rising.

One of preparations which are most commonly used for treatment and prevention of alopecia is one that contains minoxidil. There are two hair-regrowth agents which have received approval from the U.S. Food and Drug Administration, and minoxidil is one of those approved hair-regrowth agents. Minoxidil was originally developed as a hypertension drug for the purpose of reducing blood pressure. However, when using this drug, as a side effect, a trichogenous effect was observed and thereafter, this drug became famous as a hair-regrowth agent. Although mechanisms by which minoxidil works as a hair-regrowth agent is not clearly understood, it is inferred that minoxidil increases blood flow by expansion of blood vessels, whereby roots of hairs are supplied with more nutrition and eventually, growth of hairs are promoted.

Such a model of blood flow increase has been indirectly supported by a recent report that minoxidil enhances the expression of vascular endothelial growth factor (VEGF), a growth factor associated with vasodilatation in the dermal papilla which is a main cell making up the hair roots. Also, other than the vasodilative effect of the minoxidil in the hair-restoring mechanism, it has been reported that minoxidil promotes activation of dermal papilla cells in the roots of hair incubated in vitro, and growth of hair follicles in a tissue culture of follicles in vitro. These facts indicate that minoxidil may work directly on the roots of hair as a growth factor.

In addition, finasteride, a main component of Propecia which has started to be sold by Merck, is used for treatment of alopecia. It inhibits conversion of the male hormone testosterone into dihydrotestosterone, which is a more potent male hormone than testosterone. On December of 1997, the 1 mg finasteride tablet was approved by the U.S. FDA as a hair-regrowth agent for treatment of male pattern hair loss in men only, and is now commercially available. In clinical studies, it has been demonstrated to have a significant trichogenous effect. However, there has been a report that finasteride may inhibit male sexual function as a side effect. Since neither finasteride nor minoxidil show superior effect in clinical tests, and there is concern about side effects, many researches are conducted to develop a new and improved hair-regrowth agent.

The cyclosporin family of drugs has immunosuppressive activity. It is also effective to inhibit growth of virus, fungus, protozoan, etc. and has various physiological effects such as neoprotoxicity, hepatotoxicity, hypertension, enlargement of periodontium, trichogenous effect, and so on, as side effects. Cyclosporin A, a representative cyclosporin, is a cyclic peptide having the following Chemical Formula, which comprises 11 amino acids, including several N-methyl amino acids and D-alanine at No. 8 residue.

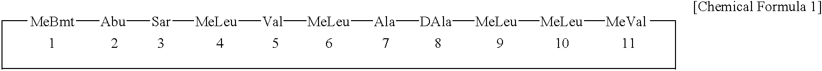

in which

MeBmt is N-methyl-(4R)-4-[(E)-2-butenyl]-4-methyl-L-threonine,

Abu is L-α-aminobutyric acid,

Sar is sarcosine,

MeLeu is N-methyl-L-leucine,

Val is L-valine,

Ala is L-alanine,

DAla is D-alanine,

MeVal is N-methyl-L-valine.

The amino acid form of cyclosporin A of the above Chemical Formula 1 is L-configuration, unless otherwise specified. The residue numbering of amino acids starts from MeBmt and proceeds clockwise, i.e. 1 for MeBmt and 11 for the last MeVal (N-methyl-L-valine) as shown in the Chemical Formula 1. The Nomenclature of cyclosporin A derivatives is practiced by describing the residue which is different from that of cyclosporin A and the position thereof. For example, a derivative in which N-methyl-L-leucine at No. 9 position in cyclosporin A is substituted with γ-hydroxy-N-methyl-L-leucine, is expressed as [γ-hydroxy-N-methyl-L-leucine⁹] cyclosporin A. Also, residues are described following commonly used abbreviations. That is, MeLeu refers to N-methyl-L-leucine, MeIle refers to N-methyl-L-isoleucine, MeVal refers to N-methyl-L-valine, MeAla refers to N-methyl-L-alanine, MeNva refers to N-methyl-L-norvaline, Leu refers to L-leucine, Ile refers to L-isoleucine, and Sar refers to sarcosine.

So far, possible development of cyclosporin as a hair-regrowth agent has been studied by many research groups. Particularly, researches involving animal hair regrowth tests, human alopecia areata (J. Am. Acad. Dermatol., 1990, 22:242-250), human male pattern alopecia (J. Am. Acad. Dermatol., 1990, 22:251-253 and Skin Pharmacol., 1994, 7:101-104), and inhibition effect of hair loss by chemotherapy in animal models (Am. J. Pathol., 1997, 150:1433-1441) have been widely conducted. In comparative experiments on mouse's back, it is shown that cyclosporin has a hair regrowth effect about 100 times superior to minoxidil Based on such findings, there have been attempts to utilize cyclosporin as a treatment for male pattern alopecia, and many applications for patents have been filed.

For example, Japanese Patent Publication Kokai Nos. Sho 60-243008, Sho 62-19512 and Sho 62-19513 disclose use of cyclosporin derivatives as a hair regrowth agent. Also, European Patent Publication No. 0414632 B1 discloses a cyclosporin derivative with modified No. 8 residue, PCT Patent Publication No. WO 93/17039 and PCT Patent Publication No. WO 00/51558 disclose isocyclosporin and immunosuppressive cyclosporin derivatives, respectively. These cyclosporins and derivatives thereof are provided as a hair regrowth agent. Furthermore, in U.S. Pat. No. 5,807,820 and U.K. Patent No. 2,218,334 A, preparations containing cyclosporins with excellent transdermal absorption are suggested for new application of a hair regrowth agent. However, the all cyclosporins used in the above documents have strong immunosuppressive ability and hence, they have limits in use for treatment of general hair loss, despite their excellent hair regrowth effect. Recently, in WO 0051558 a method for treating hair loss using nonimmunosuppressive cyclosporin derivatives is disclosed. However, the structure of [γ-hydroxy-N-methyl-L-leucine⁹]cyclosporin A claimed in the present invention is not included.

### DISCLOSURE OF THE INVENTION

Therefore, in order to find a novel hair growth promoter without problems involved in the prior art, the present inventors have examined the main metabolic products of cyclosporin for their hair growth effect, while considering their potential immunosuppressive properties. The main metabolites examined include M17, a metabolite wherein a hydroxy group is added to a η carbon of No. 1 residue, MeBmt, M21, a metabolite wherein a N-methyl group is removed from the No. 4 residue MeLeu (N-methyl-L-leucine), and M1, a metabolite wherein a hydroxy group added to a γcarbon of No. 9 residue (MeLeu). As a result, it was found that only the M1 showed an excellent hair growth effect while having reduced immunosuppressiveness. The M1 is named as [γ-hydroxy-N-methyl-L-lecine₉]cyclosporin A according to the common nomenclature, and its immunosuppressiveness is known to be lower than that of cyclosporin A (see, Transplantation 1987; 43:123-127, Clin. Chem. 1990; 36:225-229, and Transplant. Proc. 1988; 20:575-584).

Thus, the above present invention is directed to a hair growth promoter comprising, as an active ingredient, a metabolite of cyclosporin A, that is [γ-hydroxy-N-methyl-L-leucine⁹]cyclosporin A, in which a hydroxy group is added to a γ carbon of No. 9 residue MeLeu, and represented by the following formula (I):

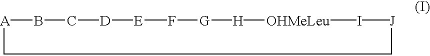

in which

A is N-methyl-(4R)-4-[(E)-2-butenyl]-4-methyl-L-threonine (MeBmt), (2S,3R,4R,6E)-3-sulfhydryl-4-methyl-2-(methylamino)-6-octenoic acid, or (2S,4R,6E)-3-oxo-4-methyl-2-(methylamino)-6-octenoic acid;

B is L-α-aminobutyric acid (Abu), L-alanine (Ala), L-threonine (Thr), L-valine (Val), or L-norvaline (Nva);

C is sarcosine, N-methyl-D-alanine ((D)—N(CH₃)—CH(CH₃)—CO—), (D)-2-(methylamino)pent-4-enoyl ((D)—N(CH₃)—CH(CH₂CHCH₂)—CO—), (D)-2-(methylamino)pent-4-ynoyl ((D—N(CH₃)—CH(CH₂CCH)—CO—), (D)-(methylthiosarcosine ((D)-Sar(2-Sme), (D)—N(CH₃)—CH(SCH₃)—CO—), N-methyl-D-serine ((D)—N(CH₃)—CH(CH₂OH)—CO—), (D)-2-(methylamino)butanoyl ((D)—N(CH₃)—CH(CH₂CH₃)—CO—), N-methyl-D-norvaline ((D)—N(CH₃)—CH(CH₂CH₂CH₃)—CO—), (D)-2-(methylamino)hex-4-ynoyl ((D)—N(CH₃)—CH(CH₂CCH₃)—CO—) or O-propenyl-N-methyl-D-serine ((D)—N(CH₃)—CH(CH₂OCH₂CHCH₂)—CO—);

D is N-methyl-L-leucine, γ-hydroxy-N-methyl-L-leucine, or L-valine;

E is L-valine, or L-norvaline;

F is N-methyl-L-leucine, γ-hydroxy-N-methyl-L-leucine, or L-leucine;

G is L-alanine or L-α-aminobutyric acid;

H is D-alanine or D-serine,

OHMeLeu is γ-hydroxy-N-methyl-L-leucine;

I is N-methyl-L-leucine, γ-hydroxy-N-methyl-L-leucine, or L-leucine; and

J is N-methyl-L-valine or L-valine.

The preferred metabolites of cyclosporin of the above Chemical Formula 1 having hair regrowth activity are compounds, [γ-hydroxy-N-methyl-L-leucin⁹]cyclosporin A, represented by the following formula (II).

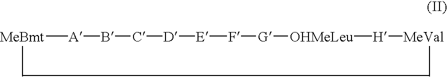

in which

MeBmt is N-methyl-(4R)-4-[(E)-2-butenyl]-4-methyl-L-threonine,

A′ is L-α-aminobutyric acid (Abu), L-alanine (Ala), L-threonine (Thr), L-valine (Val), or L-norvaline (Nva);

B′ is sarcosine, N-methyl-D-alanine ((D)—N(CH₃)—CH(CH₃)—CO—), D-2-(methylamino)pent-4-enoyl ((D)—N(CH₃)—CH(CH₂CHCH₂)—CO—), (D)-2-(methylamino)pent-4-ynoyl ((D)—N(CH₃)—CH(CH₂CCH)—CO—), or D-methylthiosarcosine (D-Sar(2-Sme), (D)—N(CH₃)—CH(SCH₃)—CO—), N-methyl-D-serine ((D)—N(CH₃)—CH(CH₂OH)—CO—), (D)-2-(methylamino)butanoyl ((D)—N(CH₃)—CH(CH₂CH₃)—CO—), N-methyl-D-Norvaline ((D)—N(CH₃)—CH(CH₂CH₂CH₃))—CO—), (D)-2-(methylamino)hex-4-ynoyl ((D)—N(CH₃)—CH(CH₂CCH₃)—CO—), or O-propenyl-N-methyl-D-serine ((D)—N(CH₃)—CH(CH₂OCH₂CHCH₂)—CO—);

C′ is N-methyl-L-leucine, γ-hydroxy-N-methyl-L-leucine, or L-valine;

D′ is L-valine or L-norvaline;

E′ is N-methyl-L-leucine, γ-hydroxy-N-methyl-L-leucine, or L-leucine;

F′ is L-alanine or L-α-aminobutyic acid;

G′ is D-alanine or D-serine;

OHMeLeu is γ-hydroxy-N-methyl-L-leucine;

H′ is N-methyl-L-leucine, γ-hydroxy-N-methyl-L-leucine, or L-leucine; and

MeVal is N-methyl-L-valine.

The more preferred [γ-hydroxy-N-methyl-L-leucine⁹]cyclosporin A of the above Chemical Formula 1 having hair regrowth activity are compounds represented by the following formula (III).

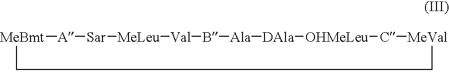

in which

MeBmt is N-methyl-(4R)-4-[(E)-2-butenyl]-4-methyl-L-threonine,

A″ is L-α-aminobutyric acid (Abu), L-alanine (Ala), L-threonine (Thr), L-valine (Val), or L-norvaline (Nva);

Sar is sarcosine;

MeLeu is N-methyl-L-leucine;

Val is L-valine;

B″ is N-methyl-L-leucine, or L-leucine;

Ala is L-alanine;

DAla is D-alanine;

OHMeLeu is γ-hydroxy-N-methyl-L-leucine;

C″ is N-methyl-L-leucine or L-leucine; and

MeVal is N-methyl-L-valine.

The even more preferred [γ-hydroxy-N-methyl-L-leucine⁹]cyclosporin A of the above Chemical Formula 1 having hair regrowth activity are compounds represented by the following formula (IV).

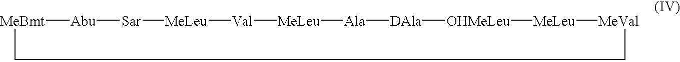

in which

MeBmt is N-methyl-(4R)-4-[(E)-2-butenyl]-4-methyl-L-threonine,

Abu is L-α-aminobutyric acid;

Sar is sarcosine;

MeLeu is N-methyl-L-leucine;

Val is L-valine;

DAla is D-alanine;

OHMeLeu is γ-hydroxy-N-methyl-L-leucine; and

MeVal is N-methyl-L-valine.

In another aspect, the present invention is directed to a liquid formulation, spray, gel, paste, emulsion, cream, conditioner, or shampoo formulated from the composition comprising [γ-hydroxy-N-methyl-L-leucine⁹]cyclosporin A as an active ingredient having a hair growth promoting effect.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a result of a High Pressure Liquid Chromatography of [γ-hydroxy-N-methyl-L-leucine⁹]cyclosporin A;

FIG. 2 is a ¹H-NMR spectrum of [γ-hydroxy-N-methyl-Lleucine⁹]cyclosporin A;

FIG. 3 is a ¹³C-NMR spectrum of [γ-hydroxy-N-methyl-L-leucine⁹]cyclosporin A;

FIG. 4 is a photograph of a control group in the animal test measuring hair growth effects of cyclosporin A and [γ-hydroxy-N-methyl-L-leucine⁹]cyclosporin A using C57BL/6 mice;

FIG. 5 is a photograph of a group treated with cyclosporin A in the animal test measuring hair growth effects of cyclosporin A and [γ-hydroxy-N-methyl-L-leucine⁹]cyclosporin A using C57BL/6 mice; and

FIG. 6 is a photograph of a group treated with [y-hydroxy-N-methyl-L-leucine⁹]cyclosporin A in the animal test measuring hair effects of cyclosporin A and [γ-hydroxy-N-methyl-L-leucine⁹]cyclosporin A using C57BL/6 mice.

### BEST MODE FOR CARRYING OUT THE INVENTION

The present invention is described in detail as follows. In order to develop a novel hair regrowing agent, the present inventors produced various metabolites of cyclosporin and carried out the hair regrowth evaluation tests for the metabolites. As a result, it was found that [γ-hydroxy-N-methyl-L-leucine⁹]cyclosporin A has an superior hair regrowth (restoring) effect than any other compounds.

The following examples are given by way of illustration of the best mode contemplated by the inventor(s) of carrying out the invention. However, those skilled in the art will appreciate that various modifications, additions and substitutions are possible, without departing from the scope and spirit of the invention.

### Reference Example

### Reference Example 1

### Preparation of the Metobolite M21 ([Leu⁴]cycolosporin A)

Decapeptide (H-Val-MeLeu-Ala-(D)Ala-MeLeu-MeLeu-MeVal-MeBmt(Ac)-Abu-Sar-Ome) was condensed with Boc-Leu-OH using condensing agents of benzotriazol-1-yl-oxy-tris-(dimethylamino)-phosphonium hexafluorophosphate) and dimethylaminopyridine. The undecapeptide thus obtained was deprotected using sodium hydroxide (NaOH) and trifluoroacetic acid(TFA). The product was then subjected to a cyclization reaction using benzotriazol-1-yl-oxy-tris-(dimethylamino)-phosphonium hexafluorophosphate and dimethylaminopyridine to form a substituted cyclosporin A-acetate, which was treated with sodium methoxide (NaOMe) to remove acetyl groups. In this way, the metabolite M21, [Leu⁴]cyclosporin A, wherein a methyl group is removed from the No. 4 N-methyl-L-leucine was produced. M21 was found to have no hair growth effect as shown in an experiment according to Test Example 1.

### Reference Example 2

Preparation of the metabolite M17 (a metabolite wherein a hydroxy group is added to a carbon of No. 1 MeBmt)

The hydroxy group at No. 1 position in cyclosporin A was reacted with acetic anhydride to synthesize [O-acetyl] 1 cyclosporin A. The product was refluxed with N-bromosuccinimide in the presence of a catalyst of azobisisobutyronitrile to synthesize [O-acetyl-6-bromo] 1 cyclosporin A. The product was added to a solvent of ethyl methyl ketone and heated in the presence of a catalyst mixture of tetrabutylammonium acetate and sodium iodide to synthesize [6-acetoxy-O-acetyl] 1 cyclosporin A. The product was deacetylated with 0.5M sodium methoxide to synthesize M17. The resulting M17 was identified by Mass spectroscopy and NMR spectroscopy alalyses. M17 was found to have no hair growth effect as shown in an experiment according to Test Example 1 (J. Org. Chem. 1992; 57: 2689-2691).

### EXAMPLE 1

### Preparation of [γ-Hydroxy-N-Methyl-L-Leucine⁹]cyclosporin A

In this example, preparation of [γ-hydroxy-N-methyl-L-leucine⁹]cyclosporin A showing hair regrowth effect after being transformed by microorganisms will be described.

Psedonocardia autotrophica KCTC 9441 was used as a strain for preparing the metabolite of cyclosporin A. The strain was cultured in a medium containing 0.7% glucose, 0.45% yeast extract, 0.5% malt extract, 1.0% soluble starch and 0.005% CaCO₃ at a culturing temperature of 27° C.

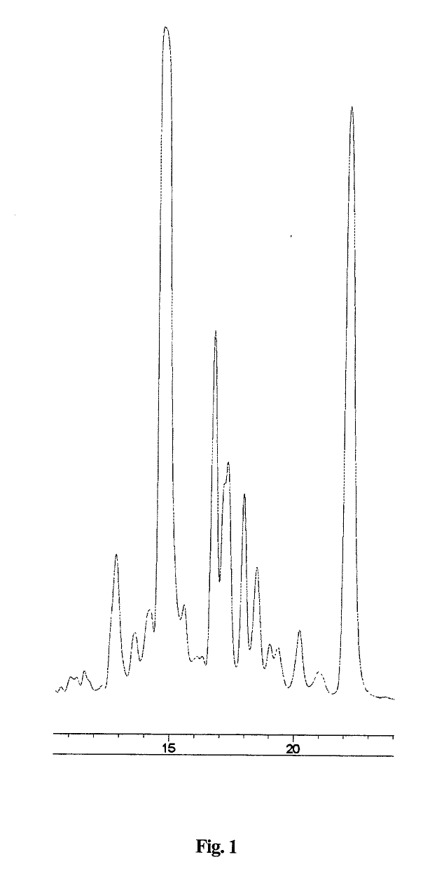

Using a fermentor for culture of the strain, a preculture period of 4 days in an Erlenmeyer flask was arranged before beginning the actual culture. The actual culture was performed in a 4 l fermentor using the above-described medium. At 24 hour after the actual culture started, cyclosporin A dissolved in methanol was added to a concentration of 100 mg/l and culturing was continued for a further 72 hours. At this time, the culture medium was extracted with ethylacetate in an amount equivalent to the entire medium, and the organic phase was concentrated. The concentrate was separated and fractionated by liquid chromatography. The liquid chromatography elution profile showing cyclosporin derivatives is shown in FIG. 1. In FIG. 1, the peak observed at 22 to 23 minutes of retention time corresponds to cyclosporin A and the peak at 15 minutes corresponds to [γ-hydroxy-N-methyl-L-leucine⁹]cyclosporin A.

Here, the derivatives were separated on a C-18 column flowing a varying mixture of solvent A and solvent B. Firstly, the concentration of a solvent A was kept at 100% for 2 minutes, reduced to 60% by 4 minutes, slowly reduced to 39% by 60 minutes and returned to 100% by 65 minutes. The solvent A was 25% aqueous methanol solution and the solvent B was 100% acetonitrile.

Also, the [γ-hydroxy-N-methyl-L-leucine⁹]cyclosporin A can be prepared using microsomal enzyme from rabbit liver.

Firstly, liver of a New Zealand White rabbit was removed and dipped in 0.1 M potassium phosphate buffer solution for 5 minutes. Chopped liver tissue was ground with a homogenizer and centrifuged (9000 g, 4°, 20 minutes). The supernatant was separated and again centrifuged (10,500 g, 1 hour). The supernatant was decanted and remaining pellet was dissolved in 0.1 M phosphate buffered saline. The resulting solution was used as an enzyme source. The prepared microsomal enzyme (50 mg), cyclosporin (1 mg) and NADPH (5 mM) were added to distilled water of an appropriate amount and reacted in a thermostatic bath set to 37° C. for 1 hour. The reaction was extracted with an equal volume of ethylacetate and analyzed.

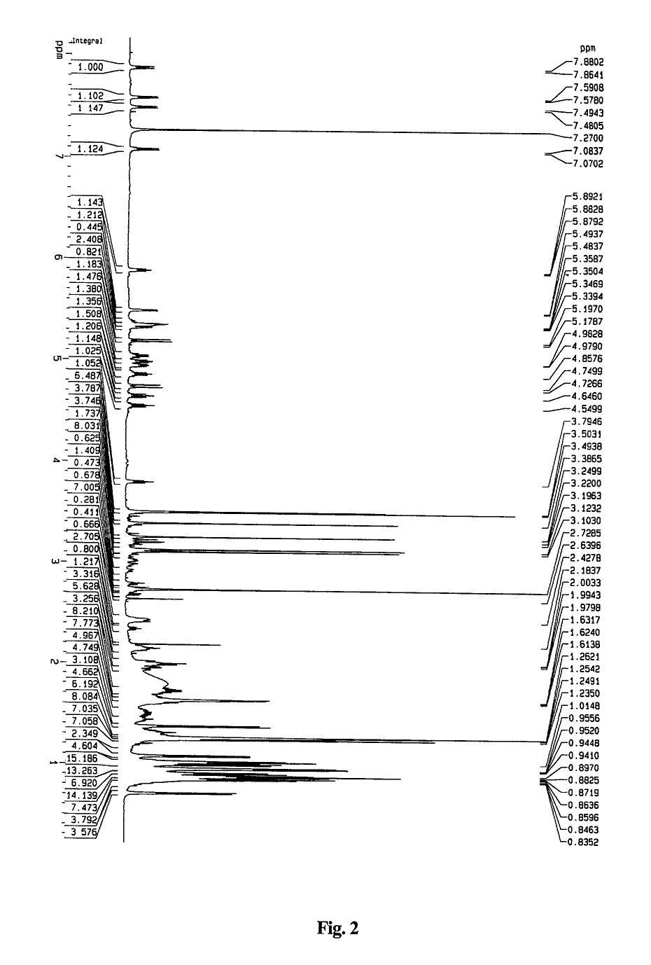

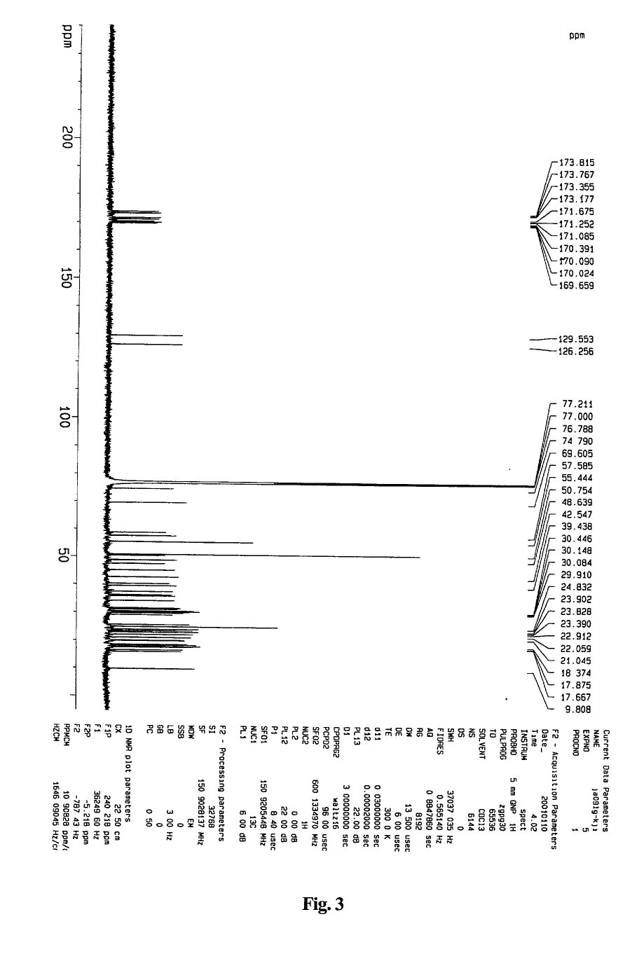

### Analysis of Structure of [γ-Hydroxy-N-Methyl-L-Leucine⁹]cyclosporin A

[γ-hydroxy-N-methyl-L-leucine⁹]cyclosporin A (C₆₂H₁₁₁N₁₁O₁₃) was analyzed according to FAB MS (ZMS AX 505H) and a peak was observed at m/z 1219 [M+H]⁺, which indicates that an oxygen atom was added to the molecule. Also, the structure of the product was identified using ¹H-NMR (Bruker NMR 600 MHz)(FIG. 2) and ¹³C-NMR (Bruker NMR 150 MHz) (FIG. 3).

### Formulations

### Formulation 1

### Preparation of a Hair Revitalizing Tonic Containing [γ-Hydroxy-N-Methyl-L-leucine⁹]cyclosporin A

Three hair revitalizing tonics as described in Table 1 below were prepared. The tonics were examined for the hair regrowth effect in animal models according to Test Example 1 described later. In the animal test, it was shown that Composition 1 has hair regrowth effect comparable to a hair revitalizing tonic containing 0.1% cyclosporin A.

TABLE 1

| Ingredients | Composition 1 | Composition 2 | Composition 3 |
|---|---|---|---|
| Ethanol | 40.0 | 40.0 | 40.0 |
| [γ-hydroxy-N-methyl- | 0.1 | 1.0 | 8.0 |
| L-leucine⁹] | | | |
| cyclosporin A | | | |
| Tocopherol acetate | 0.1 | 0.1 | 0.1 |
| Salicylic acid | 0.3 | 0.3 | 0.3 |
| L-menthol | 0.3 | 0.3 | 0.3 |
| Tween 20 | 0.5 | 0.5 | 0.5 |
| Fragrance | Prop. Amount | Prop. Amount | Prop. Amount |
| Color | Prop. Amount | Prop. Amount | Prop. Amount |

| | |
|---|---|
| Water | q.s. to 100 wt % |

### Formulation 2

### Preparation of a Hair Cream Containing [γ-Hydroxy-N-Methyl-L-Leucine⁹]cyclosporin A

Three hair creams as described in Table 2 below were prepared. Oil phase ingredients were mixed and heated to 80□ so that the ingredients formed a homogenous mixture. Separately, aqueous phase ingredients were mixed and heated to 80° C. so that the ingredients formed a homogenous mixture. The prepared two mixtures of different phases at 80° C. were combined and emulsified. The resulting emulsion was then cooled to room temperature and fragrance and colorant were added thereto to form a hair cream. At this stage, water was added to make up the volume of the hair cream.

The resulting hair creams were examined for their hair regrowth effect in animal models according to Test Example 1 described later. In the animal test, it was shown that Composition 1 described in Table 2, which contains 0.1% [γ-hydroxy-N-methyl-L-leucine⁹]cyclosporin containing 0.1% cyclosporin A.

TABLE 2

| Ingredients | Composition 1 | Composition 2 | Composition 3 |
|---|---|---|---|
| Paraffin | 5.0 | 5.0 | 5.0 |
| Setostearylalcohol | 5.5 | 5.5 | 5.5 |
| Petrolatum | 5.5 | 5.5 | 5.5 |
| Glycerine- | 3.0 | 3.0 | 3.0 |
| monostearate | | | |
| Polyoxyethylene | 3.0 | 3.0 | 3.0 |
| octyldodecylether | | | |
| Propylparaben | 0.3 | 0.3 | 0.3 |
| [γ-hydroxy-N-methyl- | 0.1 | 1.0 | 8.0 |
| L-leucine⁹] | | | |
| cyclosporin A | | | |
| Glycerin | 7.0 | 7.0 | 7.0 |
| Dipropyleneglycol | 20.0 | 20.0 | 20.0 |
| Polyethyleneglycol | 5.0 | 5.0 | 5.0 |
| Water | q.s. to 100 wt % without fragrance and colorant | | |
| Fragrance | Prop. Amount | Prop. Amount | Prop. Amount |
| Colorant | Prop. Amount | Prop. Amount | Prop. Amount |

### Formulation 3

### Preparation of a Shampoo Containing [γ-Hydroxy-N-Methyl-L-Leucine⁹]cyclosporin A

Three shampoos as described in Table 3 below were prepared. Ingredients except for the fragrance, colorant and water were mixed and heated while being stirred so that the ingredients formed a homogenous mixture. The resulting mixture was then cooled to room temperature and fragrance and colorant were added thereto. Finally, water was added to make up the volume of the shampoo.

TABLE 3

| Ingredients | Composition 1 | Composition 2 | Composition 3 |
|---|---|---|---|
| Sodium POE | 40.0 | 40.0 | 40.0 |
| laurylsulfuric acid | | | |
| (30 wt % aqueous | | | |
| solution) | | | |
| Palm oil fatty acid | 3.0 | 3.0 | 3.0 |
| Diethanolamide | | | |
| propylene glycol | 2.0 | 2.0 | 2.0 |
| Methyl | 0.2 | 0.2 | 0.2 |
| paraoxybenzoic acid | | | |
| Ethanol | 2.0 | 2.0 | 2.0 |
| [γ-hydroxy-N-methyl- | 1.0 | 3.0 | 10.0 |
| L-leucine⁹] | | | |
| cyclosporin A | | | |
| Salicylic acid | 0.3 | 0.3 | 0.3 |
| L-menthol | 0.3 | 0.3 | 0.3 |
| Fragrance | Prop. Amount | Prop. Amount | Prop. Amount |
| Colorant | Prop. Amount | Prop. Amount | Prop. Amount |

| | |
|---|---|
| Water | q.s. to 100 wt % |

### Formulation 4

### Preparation of a Hair Conditioner Containing [γ-Hydroxy-N-Methyl-L-leucine⁹]cyclosporin A

Three hair conditioners as described in Table 4 below were prepared. Oil phase ingredients were mixed and heated to 80° C. so that the ingredients formed a homogenous mixture. Separately, aqueous phase ingredients were mixed and heated to 80° C. so that the ingredients formed a homogenous mixture. The prepared two mixtures of different phases at 80° C. were combined and emulsified. The resulting emulsion was then cooled to room temperature and fragrance and colorant were added thereto to form a hair conditioner. Here, water was added to make up the volume of the hair conditioner.

TABLE 4

| Ingredients | Composition 1 | Composition 2 | Composition 3 |
|---|---|---|---|
| Cetanol | 3.0 | 3.0 | 3.0 |
| Self-emulsifiable | 2.0 | 2.0 | 3.0 |
| Glycerol-monostearate | | | |
| Squalene | 10.0 | 10.0 | 10.0 |
| [γ-hydroxy-N-methyl- | 1.0 | 5.0 | 10.0 |
| L-leucine⁹] | | | |
| cyclosporin A | | | |
| Propylene glycol | 2.0 | 2.0 | 2.0 |
| Stearyldimethyl | 8.0 | 8.0 | 8.0 |
| Benzylammonium | | | |
| chloride | | | |
| (25 wt % aqueous | | | |
| solution) | | | |
| Methyl | 0.2 | 0.2 | 0.2 |
| paraoxybenzoic acid | | | |
| Salicylic acid | 0.3 | 0.3 | 0.3 |
| L-menthol | 0.3 | 0.3 | 0.3 |
| Water | q.s. to 100 wt % | | |
| Fragrance | Prop. amount | Prop. amount | Prop. amount |
| Colorant | Prop. amount | Prop. amount | Prop. amount |

### Test Example 1

### Test of Hair Regrowth Effects of [γ-hydroxy-N-Methyl-L-Leucine⁹]cyclosporin A

C57BL/6 mice (female), 42˜49 days old, were used in this test.

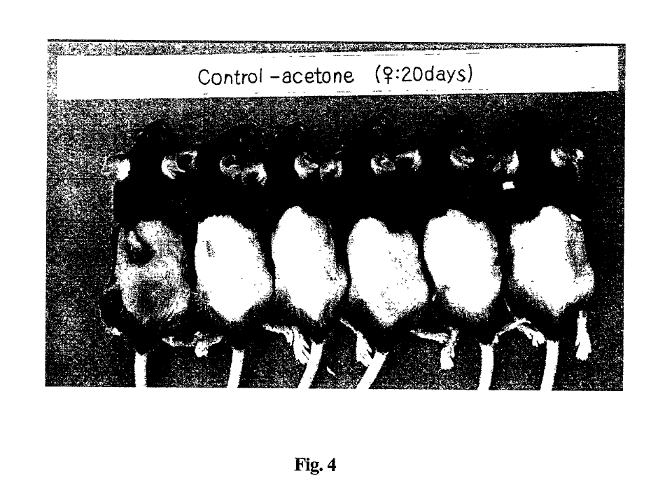

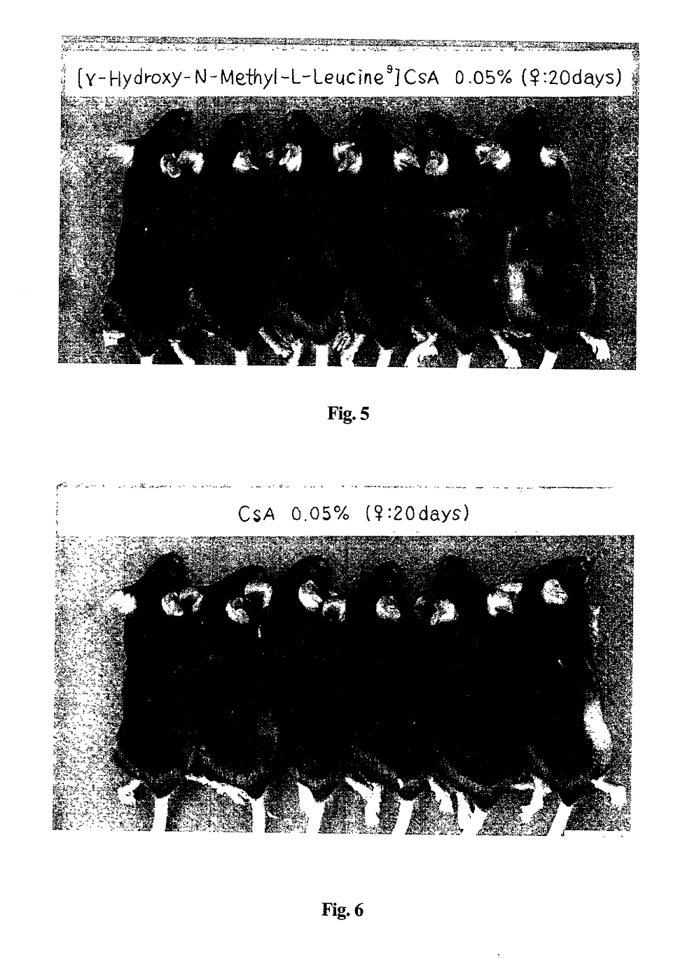

The mice were removed of hair on their backs using an electric shaver, and weighed. The mice were divided into several groups with weights equally distributed. After one day of adaptation, cyclosporin A, main metabolites of cyclosporin A, such as [γ-hydroxy-N-methyl-L-leucine⁹]cyclosporin A, M17, M21, and control were applied over the hair removed area once a day per each individual for 30 days. Here, the applied amount of cyclosporin A and metabolites thereof was 100 μl (0.05% w/v). The degree of hair growth were judged by naked eye and the back sides of the mice were photographed. FIG. 4 shows a photograph of a control group in the animal test for measuring hair growth effects of cyclosporin A and [γ-hydroxy-N-methyl-L-leucine⁹]cyclosporin A using C57BL/6 mice. FIG. 5 shows a photograph of a group treated with cyclosporin A in the test for measuring hair growth effects of cyclosporin A and [γ-hydroxy-N-methyl-L-leucine⁹]cyclosporin A using C57BL/6 mice. FIG. 6 shows a photograph of a group treated with [γ-hydroxy-N-methyl-L-leucine⁹]cyclosporin A in the test for measuring hair growth effects of cyclosporin A and [γ-hydroxy-N-methyl-L-leucine⁹]cyclosporin A using C57BL/6 mice, in which it is noted that the result is comparable to that of cyclosporin A, that is before transformation. In the mean time, metabolites M17 and M21 show no significant effect.

Upon observing the back conditions of mice during the test period of 20 days, no peculiar skin irritations were observed in the control group and all treated groups.

### Test Example 2

### Test of Immunosuppression of [γ-Hydroxy-N-Methyl-L-Leucine⁹]cyclosporin A

The test of immunosuppression was carried out using peripheral blood mononuclear cells (PBMC) obtained from healthy human adult treated with PHA (Phytohemagglutinin), a cell division stimulant, according to the MLR method (Mixed Lymphocyte Reaction method, J. Antibiotics, 1994, 47:208-215).

A group of cells (4×10⁶/ml) treated with mitomycin C (30 μg/ml, 30 min.) as stimulant cells was mixed with an equal number of untreated reactive cell group. The resulting mixture was incubated for 4 days. During the incubation, the mixture was treated with cyclosporin A and derivatives thereof to be examined including [γ-hydroxy-N-methyl-L-leucine⁹]cyclosporin A in serial dilutions from 10⁻⁶ M to 10⁻¹¹ M. After 4 days incubation, ³H-thymidine was added to the mixtures and incubated for an additional 16 hours. Then, the amount of thymidine introduced into the cells was measured (liquid scintillation counter) and IC₅₀ (μg/ml) of respective cyclosporins were calculated.

As a result, IC₅₀ (μg/ml) of cyclosporin A was found to be 0.035, 0.025 and 0.030, while [γ-hydroxy-N-methyl-L-leucine⁹]cyclosporin A was 0.165, 0.178 and 0.150. Thus, it was noted that [γ-hydroxy-N-methyl-L-leucine⁹]cyclosporin A had lower immunosuppressive effect than cyclosporin A, which accorded with the data in the literature (Transplantation 1987, 43:123-127)

Also, to examine the ability to inhibit cell proliferation against stimulation by PHA, to mononuclear cells (4×10⁶/ml) which had been treated with PHA (10 μg/ml) were added cyclosporin A and derivatives thereof including [γ-hydroxy-N-methyl-L-leucine⁹]cyclosporin A in serial dilutions from 10⁻⁶ M to 10⁻¹¹ M, followed by incubation for 3 days. Then, like in the MLR method, ³H-thymidine was added to the cells, which were again incubated for additional 16 hours. After the incubation, IC₅₀ (μg/ml) of respective cyclosporins were calculated. IC₅₀ (μg/Ml) of cyclosporin A was 0.25, 0.45 and 0.32, while [γhydroxy-N-methyl-L-leucine⁹]cyclosporin A was 1.23, 2.25 and 1.50. Thus, it was noted that [γ-hydroxy-N-methyl-L-leucine⁹]cyclosporin A had lower immunosuppressive effect than cyclosporin A.

On the basis of these results, the present compound is formulated into a form of a liquid formulation, spray, gel, paste, emulsion, cream, conditioner, or shampoo.

In the hair regrowth agent according to the present invention, the administrated amount capable of promoting hair regrowth is 0.01 to 30%, preferably 0.1 to 10% based on total weight of composition.

### INDUSTRIAL APPLICABILITY

A hair growth promoter comprising [γ-hydroxy-N-methyl-L-leucine⁹]cyclosporin A as an active ingredient according to the present invention has excellent hair growth promoting effect, leading the superior hair restoring effect while maintaining lower immunosuppression.

What is to be claimed is:

1. A hair growth promoter comprising [γ-hydroxy-N-methyl-L-leucine⁹]cyclosporin A as an active ingredient.

2. The hair growth promoter as set forth in claim 1, which is formulated in a form selected from the group consisting of liquid formulation, spray, gel, paste, emulsion, cream, conditioner, and shampoo.
